(19) United States
(12) Patent Application Publication
(10) Pub. No.: US 2017/0303814 A1
(43) Pub. Date: Oct. 26, 2017

(54) WEARABLE DEVICE FOR SKIN CONDUCTANCE MEASUREMENT

(71) Applicant: KONINKLIJKE PHILIPS N.V., EINDHOVEN (NL)

(72) Inventor: Stefan VAN DE PAS, Herten (NL)

(21) Appl. No.: 15/510,072

(22) PCT Filed: Sep. 22, 2015
(86) PCT No.: PCT/EP2015/071623

§ 371 Date: Mar. 9, 2017

(30) Foreign Application Priority Data
Sep. 30, 2014 (EP) 14186956.0

(51) Int. Cl. A61B 5/053; A61B 5/00

(52) U.S. Cl. CPC A61B 5/0531; A61B 5/681; A61B 5/7203

(57) ABSTRACT

The present invention relates to a device for skin conductance measurement in a frequency range up to at least 50 Hz. The proposed device comprises two measurement terminals (23, 24) for applying a constant DC voltage to a skin area, a first measurement path (27) coupled between a first of said measurement terminals (23) and a first output terminal (25), a second measurement path (28) coupled between the second measurement terminals (24) and a second output terminal (26), two output terminals (25, 26) each providing a respective measurement voltage, the difference of which being related to the skin conductance of said skin area. This provide for cancelation of noise when measuring the difference at the output terminals.

### FIELD OF THE INVENTION

The present invention relates to a wearable device for skin conductance measurement in a frequency range up to at least 50 Hz.

### BACKGROUND OF THE INVENTION

Different ways of measuring electro dermal activity (EDA) are known, for instance using the exosomatic DC measurement in an analog circuit as e.g. described in Wolfram Boucsein, “Electrodermal Activity”, Springer Science & Business Media, Feb. 2, 2012. The most commonly used DC methods are called the quasi-constant method and the quasi-constant voltage method, both applying a voltage divider method. For instance, with the quasi-constant voltage method a constant voltage is applied to the skin and the conductance is measured. Often a voltage of 0.5V is applied and a standard location of the electrodes is used. A high impedance amplifier is used for measurement of the voltages in the circuit.

These voltage divider measurements, in particular the quasi-constant voltage methods, have a number of disadvantages. The dynamic range of a skin conductance limited resistor used in the voltage divider, needs to be small compared to the skin resistance since otherwise a majority of the voltage will not be across the skin. Further, thermal noise tends to be high because the skin resistance may be high e.g. 1 nano Siemens. Still further, circuits are susceptible for capacitive 50/60 Hz EMI coupling via the electrodes on the skin, if the frequency band of interest is up to 50 Hz or even up to 100 Hz.

### SUMMARY OF THE INVENTION

It is an object of the present invention to provide a wearable device for skin conductance measurement in a frequency range up to at least 50 Hz, preferably up to 100 Hz, which overcomes the disadvantages of the known devices.

In a first aspect of the present invention a wearable device for skin conductance measurement in a frequency range up to at least 50 Hz is presented, said device comprising:

two measurement terminals for applying a constant DC voltage to a skin area,

a first measurement path coupled between a first of said measurement terminals and a first output terminal,

a second measurement path coupled between the second measurement terminals and a second output terminal,

the first and second output terminals each providing a respective measurement voltage, the difference of which being related to the skin conductance of said skin area, wherein each of said first and second measurement paths comprises an identical resistance circuit.

Usually skin conductance measurement deals with very low frequency (<4 Hz) information; however, the proposed skin conductance measurement desires to provide a much larger bandwidth of up to at least 50 Hz, preferably up to 100 Hz. Since in this frequency band 50/60 Hz capacitive coupling to the measurement circuit via the electrodes (that are coupled to the measurement terminals of the proposed device when used in practical operation) is unavoidable, the circuit is extended to allow the electrodes to be floating to a certain extent to the circuit ground. The characteristic of the capacitive coupling is that it is common for both electrodes. Provided that the 50/60 Hz noise is coupled symmetrically to both electrodes the signal can be cancelled by subtracting the outputs of both measurement paths as is e.g. done with analog-to-digital conversion (ADC) with a differential input.

Further, since the proposed skin conductance measurement is intended to be integrated in a wearable device like a watch, the location of the electrodes is different (they are e.g. positioned on the wrist) compared to the known devices and the excitation voltage is also increased to 1.024V. Research has shown that a suitable response can be measured in this way.

Each of said first and second measurement paths comprises a resistance circuit, preferably an identical resistance circuit. This improves and simplifies the measurement of the skin conductance since the undesired noise, which is equally present in both measurement paths, is cancelled out quite accurately in this way. Hence, cancelation of noise (EMI) is provided when measuring the difference between the output terminals.

In an embodiment the proposed device further comprises a pair of electrodes, wherein to each of said measurement terminals one of said electrodes is coupled. This enables the desired skin conductance measurement via the electrodes that may be appropriately arranged at the subject. The kind of electrodes is generally not of importance; various kinds of electrodes usable for this purpose are generally known in the art.

Still further, in an embodiment said first and second measurement paths are configured to apply a DC voltage in the range of 0.1 to 5V, in particular in the range of 0.5 to 1.5 V, to said measurement terminals.

Preferably, each of said first and second measurement paths comprises a low pass filter unit, in particular a low pass filter formed by a parallel coupling of a resistor and a capacitor. The low pass filters reduce bandwidth and therefore thermal noise and avoid aliasing.

In a preferably implementation each of said first and second measurement paths comprises an operational amplifier. This provides that the feedback circuit guarantees a constant voltage over the skin.

### BRIEF DESCRIPTION OF THE DRAWINGS

These and other aspects of the invention will be apparent from and elucidated with reference to the embodiment(s) described hereinafter. In the following drawings

FIG. 1 shows a schematic diagram of a known skin conductance module,

FIG. 2 shows a circuit diagram of a known device for skin conductance measurement,

FIG. 3 shows a circuit diagram of an embodiment of a device for skin conductance measurement according to the present invention,

FIG. 4 shows a circuit diagram illustrating the thermal noise contribution of the skin conductance,

FIG. 5 shows a circuit diagram illustrating the thermal noise contribution of another noise source,

FIG. 6 shows a circuit diagram illustrating the thermal noise contribution of an operational amplifier,

FIG. 7 shows a circuit diagram illustrating the thermal noise contribution of another noise source and

FIG. 8 shows a simplified circuit diagram of an embodiment of a device according to the present invention.

### DETAILED DESCRIPTION OF THE INVENTION

FIG. 1 shows a schematic diagram of a known skin conductance module 1 for skin conductance measurement. The skin conductance module 1 is generally a module that interfaces with a host system and delivers processed skin conductance measurements. Via the electrodes 2, 3 the module 1 interfaces to the skin of a subject, e.g. of a patient. The skin is schematically represented here by the resistor Rₛₖᵢₙ, illustrating also that the conductance of the part of the skin between the electrodes 2, 3 shall be measured.

The function of analog circuit 4 is to perform the measurement of electro dermal activity (EDA) using e.g. the exosomatic DC measurement. The most commonly used DC method, called the constant voltage method, may e.g. be used. With this method a constant voltage is applied to the skin and the conductance is measured. Conventionally, often a voltage of 0.5V is applied and a standard location (e.g. the palm or the volar surfaces of the fingers, or other locations as described in the above mentioned book of Wolfram Boucsein, chapter 2.2.1.1) of the electrodes is used.

The analog-to-digital converter (ADC) 5 digitizes the measurement (i.e. makes it time discrete and level discrete). The voltage reference unit 6 provides an accurate reference voltage for the ADC 5 and excitation voltage for the skin. The microcontroller 7 provides for post processing of the measurements.

Since according to the present invention the skin conductance device is intended to be integrated in a wearable device, like a watch, heart rate monitor or wristband, the location of the electrodes is different compared to the known module, they are preferably positioned on the wrist and the excitation voltage is preferably increased to 1.024V, which provides the advantage that a suitable response can be measured in this way.

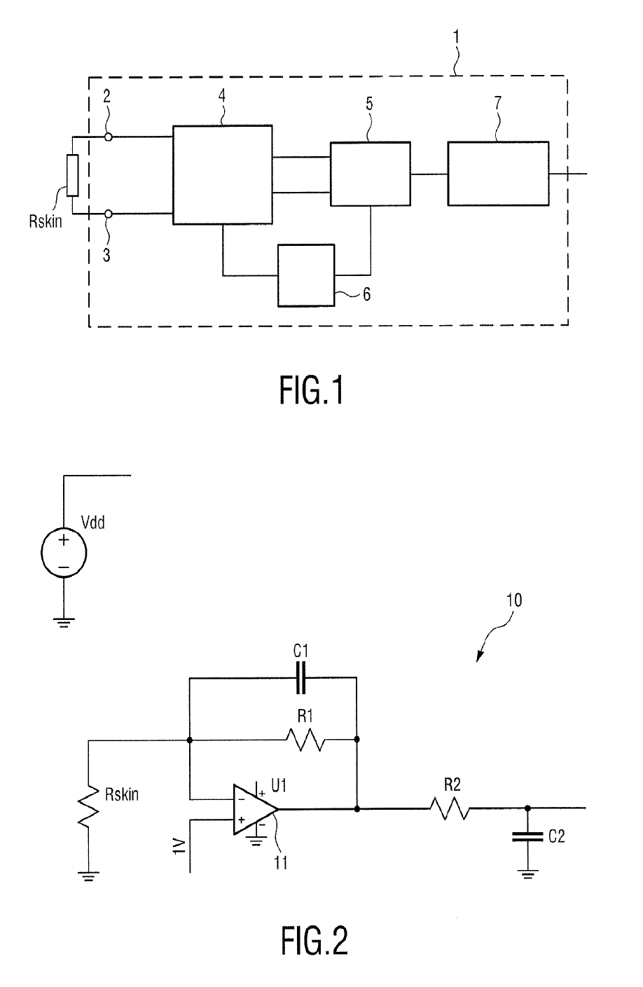

FIG. 2 shows a circuit diagram of a known device 10 for skin conductance measurement, said device 10 representing substantially the analog circuit 4 in the module 1 shown in FIG. 1. Via the virtual earth the voltage applied to the skin is kept at 1.024V. The current that flows through Rₛₖᵢₙ also flows through R1 and generates a voltage at the output of the operational amplifier 11 assuming that the current through R2 is negligible:

$$\begin{matrix}{V}_{out = \frac{1 {R}_{1}}{{R}_{skin}} = {R}_{1} \cdot {G}_{skin}} & ( 1 ) \\ {G}_{skin} = \frac{{V}_{out}}{{R}_{1}} & ( 2 )\end{matrix}$$

Hence, the output voltage of the circuit 10 is proportional the skin conductivity Gskin as reflected by equation (2). The capacitor C1 and resistor R1 as well as the capacitor C2 and the resistor R2 form two additional first order low pass filters as will be explained below.

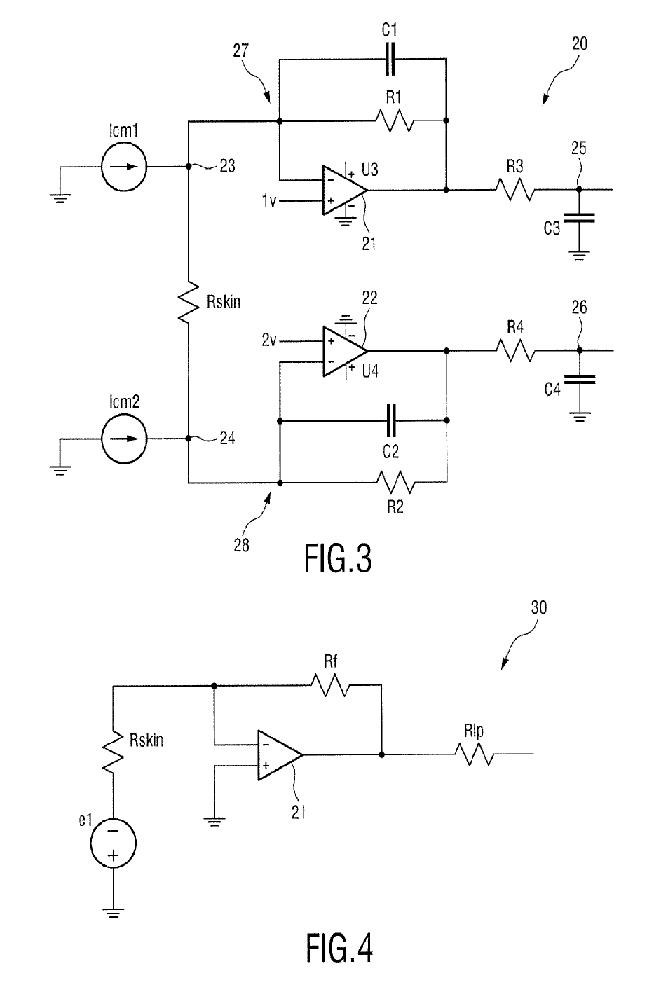

Usually, skin conductance measurement deals with very low frequency (<4 Hz) information; however, the skin conductance module requires a much larger bandwidth of 100 Hz. Since in this band 50/60 Hz capacitive coupling to the circuit via the electrodes is unavoidable, the circuit is extended to allow the electrodes to be floating to a certain extent to the circuit ground. The characteristic of the capacitive coupling is that it is common for both electrodes. A circuit diagram of a corresponding embodiment of a device 20 according to the present invention is shown in FIG. 3. The device 20 represents substantially the analog circuit 4 in the module 1 shown in FIG. 1. The device 20 comprises two input terminals (23, 24) and two output terminals (25, 26). Common coupling to both electrodes (2, 3; not shown in FIG. 3), which can be coupled to the input terminals (23, 24) is modeled as two current sources I_{cm1} and I_{cm2}. The resistor Rₛₖᵢₙ is connected in between the two input terminals (23, 24). The output terminals (25, 26) can be coupled to the AD converter (ADC) 5. Two measurement paths (27, 28) are formed between the input terminal 23 and output terminal 25, and between the input terminal 24 and the output terminal 26, respectively. The measurement path 27 comprises an operational amplifier 21 and a resistance circuit R3. The measurement path 28 comprises an operational amplifier 22 and a resistance circuit R4. The resistance values of R3 and R4 are equal. The output terminals (25, 26) each provides a respective measurement voltage. The difference of the two respective measurement voltages is related to the skin conductance between the electrodes 2, 3 that shall be measured, namely resistor Rₛₖᵢₙ. Provided that the 50/60 Hz noise is coupled symmetrically to both electrodes (2, 3), the undesired noise signal can be cancelled by subtracting the outputs at output terminals 25, 26 of both operational amplifiers 21, 22 included in the two measurement paths 27, 28 as is done with the AD converter 5 (see FIG. 1) with a differential input.

To avoid aliasing and reduce noise, capacitor C1 and resistor R1 as well as capacitor C2 and resistor R2 form two first order low pass filters by parallel coupled to the operational amplifiers 21 and 22, respectively. Filter C1/R1 will be effective as long as the operational amplifier 21 is able to maintain the virtual earth at frequencies beyond that only the filter R2/C2 will be effective. The corner frequencies are:

$${f}_{c} = \frac{1}{2 π RC} \qquad ( 3 )$$

When using a sigma-delta converter the aliasing filter requires sufficient suppression at the modulator frequency, that is 128 kHz at sample rates from 40 . . . 160 samples per second (SPS) and 32 kHz at sample rates from 5 . . . 20 SPS. If fc is around 100 Hz and the operational amplifiers U1 and U2 (21, 22) have sufficient gain-bandwidth product (GBWP) (e.g. 2 MHz) the expected suppression is for 3 decades (100 Hz . . . 100 KHz) for a second order filter this will result in 3*−40 dB=−120 dB. For a 16 bit ADC worst case at least −96 dB is needed, so fc is not very critical.

With respect to noise it holds that Vₙₚₚ of the noise at the input of the ADC should be lower than a ½ LSB:

$${V}_{npp} \leq \frac{{V}_{range}}{{2}^{N + 1}} \qquad ( 4 )$$

In this equation V_{range} is the voltage range of the ADC and N indicates the number of bits of the ADC. Supposing that the noise output signals of the operational amplifiers 21, 22 are equal, then subtraction adds a factor of √2 (i.e. sqrt(2)) to the noise, which means that the noise requirement at the output of the operational amplifiers 21, 22 is:

$${V}_{npp} \leq \frac{{V}_{range}}{{2}^{N + 3 / 2}} \qquad ( 5 )$$

Thermal RMS voltage noise over a specific bandwidth given the noise density can be calculated by:

Vₙₚₚ=6V_{RMS}=6N_{d}\sqrt{Δf} (6)

To convert to peak-peak voltage often ±3σ is used. For a resistor R the N_{d} is given by

N_{d}=\sqrt{4k_{B}TR} (7)

In this formula T is the temperature in Kelvin (often 300K is used) and k_{b} the Boltzmann constant. The range of temperatures in which skin conductance measurement can be used is 0° C. to 50° C. (or 273K to 323K). For the noise analysis 300K is used, which means that actual noise at 323K can be slightly higher (i.e. √323/√300−1=3.7% higher).

To analyze the noise at the output of the operational amplifier, the individual contributions of the noise source are analyzed. The sources can be superimposed to determine the total noise at the output.

The thermal noise contribution of Rₛₖᵢₙ is illustrated in FIG. 4 depicting a circuit diagram of a circuit 30 illustrating the thermal noise contribution of the skin conductance on one measurement path of the circuit 20 shown in FIG. 3. It holds:

$${e}_{out} = {e}_{1} \frac{{R}_{f}}{{R}_{skin}} \qquad ( 8 )$$

In this equation e1 is the thermal noise of Rₛₖᵢₙ. Since e1 increases with the square root of the resistor value this contribution gets more relevant when Rₛₖᵢₙ gets in the order of Rf. This is when the skin conductivity Gₛₖᵢₙ is in a high range (e.g. above 8μ Siemens).

The thermal noise contribution of Rf is illustrated in FIG. 5 depicting a circuit diagram of a circuit 40 illustrating the thermal noise contribution of Rf on one measurement path of the circuit 20 shown in FIG. 3. It holds:

eₒᵤₜ=e₂ (9)

In this equation e₂ is the thermal noise of Rf which would be quite low because of the relative low value and small bandwidth.

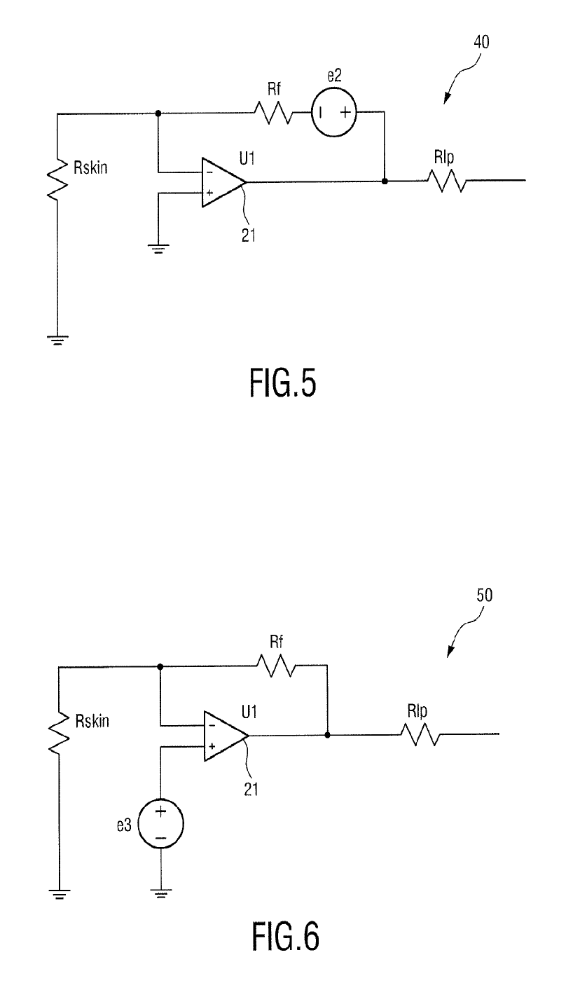

The contribution of the noise source at the positive input of the operational amplifier 21, 22 is illustrated in FIG. 6 depicting a circuit diagram of a circuit 50 illustrating the thermal noise contribution on one measurement path of the circuit 20 shown in FIG. 3. This noise can be a superposition of internal operational amplifier noise, being equivalent to thermal noise in combination with 1/f noise and other noise sources at that input like e.g. the noise of a voltage reference which typically consists also of equivalent thermal noise and 1/f noise. It holds:

$${e}_{out} = {e}_{3} + {e}_{3} \frac{{R}_{f}}{{R}_{skin}} \qquad ( 10 )$$

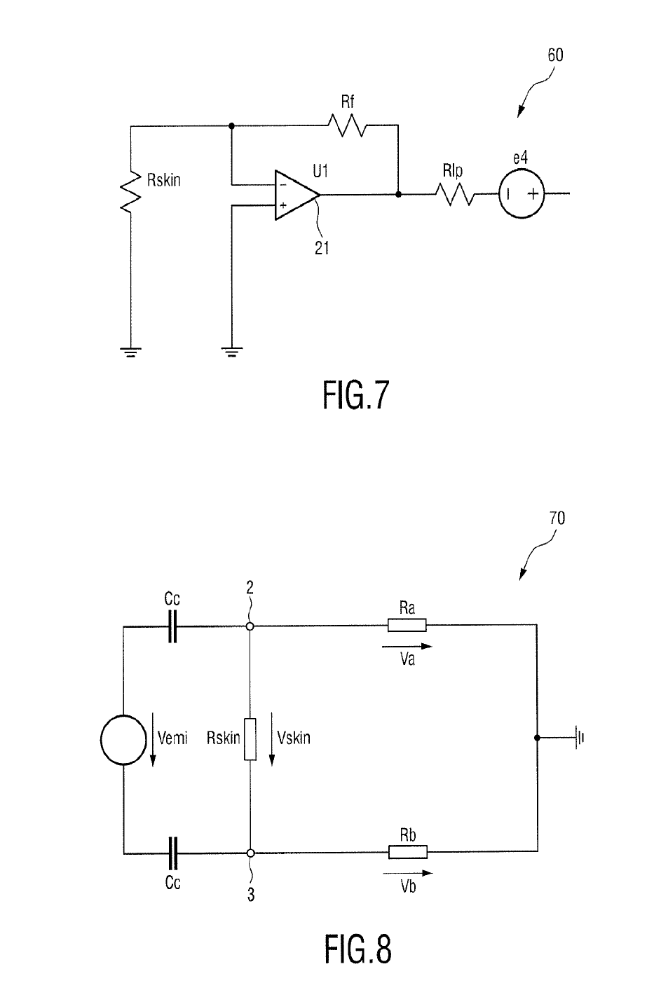

The thermal noise contribution of Rₗₚ is illustrated in FIG. 7 depicting a circuit diagram of a circuit 60 illustrating the thermal noise contribution of Rₗₚ on one measurement path of the circuit 20 shown in FIG. 3. It holds:

eₒᵤₜ=e₄ (11)

In this equation e₄ is the thermal noise of Rₗₚ which would be quite low because of the relative low value and small bandwidth.

FIG. 8 shows a simplified circuit diagram of an embodiment of a device 70 according to the present invention illustrating in simplified form the principle of the present invention. Conventionally, the circuits that measure skin conductance assume that the variation of the skin conductance as a result of constant voltage excitation on the skin that are below e.g. 10 Hz. This means that these circuits can just electrically filter any EMI (electromagnetic interference) that is picked up (typically this is 50 or 60 Hz) by the electrodes on the body by discarding frequencies higher than 10 Hz. The proposed circuit has the intention to be able to measure variations in the skin conductance up to 50 or even 100 Hz, so that electrically filtering EMI frequencies higher than 10 Hz is not an option because the EMI is in the band of interest.

Hence, referring to FIG. 8, the aim is to measure Rₛₖᵢₙ in a band of interest of e.g. up to 100 Hz. Vₛₖᵢₙ is the excitation signal of the skin, which is a DC signal, e.g. chosen as 1V, but generally in a range from 0.5 to 5V. Vₑₘᵢ and Cc form a model, i.e. Vₑₘᵢ and Cc are actually not part of the device 70, of how the EMI is injected in the circuit 70. Rₐ and R_{b} are the resistors, namely resistance circuits, in the device 70, at which Vₐ and V_{b} are measured to derive Rₛₖᵢₙ. The resistance values of Rₐ and R_{b} are equal.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

Any reference signs in the claims should not be construed as limiting the scope.

1. A wearable device for skin conductance measurement in a frequency range up to at least 50 Hz, said device comprising:
two measurement terminals for applying a constant DC voltage to a skin area,
a first measurement path coupled between a first of said measurement terminals and a first output terminal,
a second measurement path coupled between the second measurement terminals and a second output terminal,
the first and second output terminals each providing a respective measurement voltage, the difference of which being related to the skin conductance of said skin area, wherein each of said first and second measurement paths comprises an identical resistance circuit.

2. The wearable device as claimed in claim 1,
further comprising a pair of electrodes, wherein to each of said measurement terminals one of said electrodes is coupled.

3. The wearable device as claimed in claim 1,
wherein said first and second measurement paths are configured to apply a DC voltage in the range of 0.1 to 5V, in particular in the range of 0.5 to 1.5 V, to said measurement terminals.

4. The wearable device as claimed in claim 1,
wherein each of said first and second measurement paths comprises a low pass filter unit, in particular a low pass filter formed by a parallel coupling of a resistor and a capacitor.

5. The wearable device as claimed in claim 1,
wherein each of said first and second measurement paths comprises an operational amplifier.
